(19) United States
(12) Patent Application Publication
(10) Pub. No.: US 2008/0226705 A1
(43) Pub. Date: Sep. 18, 2008

(54) GEL DELIVERY SYSTEM FOR ORAL ADMINISTRATION OF MEDICAMENTS

(76) Inventors: Richard A. Soltero, Holly Springs, NC (US); Thomas J. Colatsky, Raleigh, NC (US)

Correspondence Address: MOORE & VAN ALLEN PLLC, P.O. BOX 13706, Research Triangle Park, NC, 27709, US

(21) Appl. No.: 12/062,064

(22) Filed: Apr. 3, 2008

Related U.S. Application Data
(63) Continuation-in-part of application No. 10/590,282, filed on Aug. 22, 2006.
(60) Provisional application No. 60/910,015, filed on Apr. 4, 2007.
(60) Provisional application No. 60/558,349, filed on Mar. 31, 2004.

(51) Int. Cl. A61K 9/127; A61K 9/00; A61K 47/26

(52) U.S. Cl. 424/450; 424/488; 514/781

(57) ABSTRACT

The invention relates to a swallowable delivery device fabricated from a composition including a hydrated polymeric matrix with gelatinous consistency, one or more active ingredients, and optionally one or more stiffening agent. The delivery device is of a size and shape amenable to swallowing with enhanced solubility and controlled release of the active ingredients.

### CROSS REFERENCE TO RELATED APPLICATIONS

The present invention claims priority to U.S. Provisional Application No. 60/910,015 filed on Apr. 4, 2007, and co-pending U.S. patent application Ser. No. 10/590,282 filed on Aug. 22, 2006, which in turn claims priority to PCT International Application No. PCT/US2005/009548 filed on Mar. 24, 2005, which in turn claims priority to U.S. Provisional Application No. 60/558,349 filed on Mar. 31, 2004, the contents of which are incorporated by reference herein for all purposes.

### BACKGROUND OF THE INVENTION

1. Technical Field

The present invention relates to swallowable delivery devices, and more specifically, to swallowable delivery device comprising a hydratable polymeric material impregnated with a therapeutic agent.

2. Related Art

Many medicinal drugs are administered orally as solids in tablet, capsule, pill or powder formulations. Liquid formulations of such medicines, if available, may be viable substitutions for oral solid dosage forms in refractory groups, including geriatric, pediatric and dysphagic populations, and in patients in which medicinal taste precludes dosing accuracy. Similarly, manufacturers of repurposed drugs, nutraceuticals, radio- and magnet-labeled agents require effective drug delivery strategies.

There is also an increasing demand for new technologies that enhance drug solubility. Low drug solubility often manifests itself in a numerous in vivo consequences, including decreased bioavailability, increased likelihood of food effect, increased likelihood of incomplete release from the dosage form and higher inter-patient variability. Poorly soluble compounds also present many in vitro formulation obstacles, such as severely limited choices of delivery technologies and increasingly complex dissolution testing with limited or poor correlation to the in vivo absorption.

Further, capsules currently available on the market usually include an inactive outside coating to retain a liquid active therapeutic agent encapsulated therewithin. However, these types of capsules do not provide the ability for altering the dosage because any slicing or cutting of the capsule causes the release of the therapeutic agent.

Thus, it would be advantageous to provide for the accurate delivery of soluble to insoluble drug and for oral delivery of such a drug that facilitates ingestion, swallowing, altering the dosage and/or controlled release.

### SUMMARY OF THE INVENTION

The invention relates to a swallowable delivery device fabricated from a composition comprising a hydrated polymeric matrix with gelatinous consistency, one or more active ingredients, and optionally one or more stiffening agents formed into a shape that facilitates swallowing. The composition can also include excipients and the delivery device may include an erodable enterocoating with a hydrophobic polymer. The delivery device is of a size and shape amenable to swallowing with enhanced solubility and controlled release of the active ingredients.

In one aspect, the present invention provides for a drug delivery dosage form comprising a homogenous mixture of a gelatinous or hydropolymeric compound, an active therapeutic agent and optionally a stiffening agent. Preferably, the delivery device does not include any flowable liquid or components less viscous then the gelatinous or hydropolymeric compound that forms the polymeric matrix of the delivery device.

In another aspect, the present invention provides for gelatinous or hydropolymeric delivery device for the delivery of a therapeutic agent at a controlled rate over an extended period of time, wherein the therapeutic agent is dispersed throughout a gelatinous polymeric matrix for the slow release of the therapeutic agent correlating to the dissolution of the delivery device.

In yet another aspect, the present invention provides a gelatinous or hydropolymeric delivery device exhibiting enhanced swallowability wherein the gelatinous or hydropolymeric delivery device comprises a gelatin with a bloom value from about 130 to 225 and an active therapeutic agent to form a homogeneous mixture for a controlled rate of the therapeutic agent over an extended period of time. Optionally the delivery device may further include an additional coating having a lower bloom value then the delivery device to further enhance swallowability.

In a further aspect, the present invention provides for a pharmaceutical delivery device comprising a homogenous mixture of a gelatinous compound, an active therapeutic agent and optionally a stiffening agent formed into a shape that facilitates swallowing. The formed shape is further coated with a lipid or hydrophobic polymer coating to cover any exposed therapeutic agent thereby removing any unpalatable exposure until swallowed and removed from an area having taste bud receptors.

Another aspect of the present invention provides for swallowable delivery devices comprising or consisting essentially of a hydratable polymeric material homogeneously impregnated with a therapeutic agent or active pharmaceutical agent fabricated into a shaped form sized for easy swallowability. Optionally, the swallowable delivery device is provided in a segmented form in which each segment includes a predetermined amount of the active pharmaceutical agent.

In yet another aspect, the present invention provides for a delivery device wherein the active pharmaceutical agent is soluble in the hydrated polymeric base while in other embodiments, the active pharmaceutical agent is poorly soluble. In certain embodiments in which the active pharmaceutical agent is poorly soluble, the active agent may be encapsulated in a liposome to increase solubility.

A still further aspect the present invention provides for methods of administration of the oral delivery device that comprises at least a hydratable polymeric base that is impregnated with an active pharmaceutical agent in an amount sufficient to provide a therapeutic benefit. The delivery device can be in a single unit dose, or by breaking or cutting off one or more segments to provide a unit dose, and administering the dose to a subject by oral ingestion.

Another aspect provides for a kit including a plurality of oral delivery devices, wherein each of the oral delivery devices comprise at least a hydratable polymeric base and an active pharmaceutical agent dispersed in the hydratable polymeric base in an amount sufficient to provide a therapeutic benefit. The oral delivery device may be in the form of individual unit forms or in an elongated strip of separatable dosages. The elongated strip may be in the form having marking thereon which correspond to one or dosing parameters for the pharmaceutical dosage form. Notably, because of the consistency of the elongated strip, cutting into delineated dosage strip can be easily accomplished without the loss of any therapeutic agent as evident in prior art capsules. Dosing parameters may, for example, be selected from the group consisting of age, weight, and symptom scale.

These and other aspects of the invention will be apparent from the description of the invention which follows below.

### BRIEF DESCRIPTION OF THE FIGURES

FIG. 1 shows segmented gelatinized strips including multiple dosages units in separatable forms.

FIG. 2 shows a segmented gelatinized strip having markings for delineating different dosages.

FIG. 3 shows one embodiment having an inactive core, a hydrated polymeric layer including an active therapeutic agent and a final layer of for encapsulating the active layer.

FIG. 4 shows different shaped and sized delivery devices that facilitate swallowing.

FIGS. 5 A and B shows alternative dosage shaped forms.

### DETAILED DESCRIPTION OF THE INVENTION

Definitions

As used herein, the following definitions have the meanings indicated:

As used in the specification and claims, the singular form “a”, “an” and “the” include plural references unless the context clearly dictates otherwise.

The term “oral delivery” as used herein means ingestion through the mouth by swallowing.

The term “delivery device” as used herein means a shaped form having dimensions acceptable for swallowing. The delivery device is fabricated from a therapeutic composition that is essentially homogeneous throughout the entire form, or in the alternative the delivery device includes an inert core which is coated with the therapeutic composition. Optionally an additional layer may be positioned on the therapeutic composition. The delivery device is flexible and with some elasticity to ease with swallowing.

The delivery device of the present invention, because of the gelatinous consistency of its hydrated polymeric matrix, is softly resilient, yet is appropriately firm to facilitate swallowing and passage down the esophagus without hesitation, coughing, pain, and regurgitation. It is cohesive in the mouth, and passes through the throat smoothly when swallowed. Accordingly, it is particularly suitable for medication delivery for patients with swallowing abnormalities.

The delivery device has ingestion qualities and textural properties allowing it to be readily positioned in the mouth by, e.g., pressing with the tongue, and without chewing smoothly passes through the throat. It stimulates salivation through positive enhancement of taste, smell and/or texture, which further facilitates swallowing.

The essential components in the delivery device are an active ingredient, (i.e. biologically active, therapeutic agent, medicament, plant extract, vitamin, etc.) and a hydrated polymeric material combined into an essentially homogeneous mixture. All non-active ingredient components are food grade or “generally recognized as safe” (GRAS) by those skilled in the art of pharmaceutical preparations, i.e. pharmaceutically acceptable. The delivery device can be made into a variety of shapes including a cylinder wherein its length is greater than diameter, a cylinder with flat ends, a cylinder with tapered ends, a cylinder with one tapered end, and the other end rounded or flat. The cross section of the cylinder need not be a true circle, but may be an oval or ellipse. Further, the length and diameter of the cylinder may be approximately equal. The preferred shape is a cylinder wherein its length is greater than its diameter with rounded ends.

The invention provides an improved pharmaceutical formulation for oral administration. The formulation may be used for administering a wide variety of active ingredients; and, certain embodiments are especially useful for administration to subjects who have difficulty swallowing. The active ingredients may, for example, include pharmaceutical agents, (e.g., over-the-counter, prescription generics, repurposed drugs or new chemical entities (NCEs)), vitamins, minerals, nutraceuticals, nutritional supplements, radiopharmaceuticals, magnetic drugs using magnetic microspheres as carriers and any other biologically active agent or health supplement that is suitably administered. Subjects may, for example, include pediatric, adult, and/or geriatric subjects, and may in some cases include subjects with dysphasia.

Pharmaceutical Formulations

In various aspects, the invention relates to gel like pharmaceutical formulations including a stiffener which improves the shelf life and flexibility of the formulations, pharmaceutical formulations having a segmented shape which facilitates dose adjustments, pharmaceutical formulations which mask the taste of active pharmaceutical ingredients, and pharmaceutical formulations which facilitate the delivery of poorly soluble drugs. Excipients that may enhance the physical properties of the composition to aid swallowing or drug solubility include preservatives, olfactory stimulants, salivation stimulants, solubilizing agents, pH modification agents, sweeteners, flavoring agents and antioxidants. The dosage form of the present invention possesses sufficient resiliency, textural properties and small size to facilitate comfortable swallowing. Further, those skilled in the art will recognize that the dosage form may contain or act as a sustained release formulation. Examples of such dose forms may include microencapsulated, PEGylated or other conjugated forms of the active ingredient. Each of these aspects is described in more detail in the ensuing sections.

Gelatinous Pharmaceutical Formulations with Stiffeners

The gel like formulations of the invention include a gelatinous component along with one or more stiffeners imparting a viscosity of mass to be adjusted to allow requisite flexibility without losing shape, depending on the desired sustained contact with the mouth surfaces. The gelatinous component may, for example, include hydratable polymeric materials suitable for preparation of the matrix substance described herein include materials derived from animal or vegetable proteins, such as gelatins, dextrins, soy, wheat and psyllium; gums such as acacia, guar, agar and xanthan; polysaccharides; alginates; cellulose based materials, such as, sodium carboxymethylcelluloses, hydroxypropyl methyl cellulose, hydroxypropyl cellulose, ethyl cellulose, cellulose acetate and cellulose sulphate esters; carrageenans; dextrans; pectins; synthetic polymers such as polyvinylpyrrolidone and polyacrylic acid polymers such as carboxyvinylpolymers and carbomers; and polypeptide/protein or polysaccharide complexes such as gelatin-acacia complexes.

Examples of hydratable polymers are: alginic acid or alginates, for example sodium alginate, with a viscosity of from 25 to 2000 mPa·s, a pH of about 7 and a molecular weight of about 200,000; pectin with a viscosity of from 50 to 5000 mPa·s, a pH of from 3.8 to 5.2 and a molecular weight of about 100,000; xanthan with a viscosity of from 25 to 3000 mPa·s, a pH of about 6 and a molecular weight of more than 1,000,000; galactomannan with a viscosity of from 50 to 1000 mPa·s, a pH of from 4 to 8 and a molecular weight of about 200,000; gelatin (type A or B) with a viscosity of from 5 to 200 mPa·s, a pH of from 4 to 9 and a molecular weight of from 10,000 to 100,000; sodium carboxymethylcellulose with a viscosity of from 25 to 8000 mPa·s, preferably 2500 to 8000 mPa·s, with a degree of polymerization of from 500 to 2000 and with a degree of substitution not exceeding 3, that is to say not exceeding 3, preferably 0.45 to 1.45 and particularly preferably 0.65 to 0.95 carboxymethyl groups per anhydroglucose unit; cellulose ethers with a viscosity of from 3 to 10,000 mPa·s, a degree of polymerization of from 40 to 2000 and a degree of substitution not exceeding 3, in particular hydroxyethylcellulose with a viscosity of from 3 to 10,000 mPa·s, preferably 25 to 7000 mPa·s, and a degree of substitution of about 2.5, hydroxypropyl cellulose with a viscosity of from 10 to 5000 mPa·s and a molecular weight of from 80,000 to 1,300,000 and hydroxypropyl methylcellulose with a viscosity of from 3 to 10,000 mPa·s, with a degree of substitution of from 1 to 2 and, preferably, a methoxyl content of from 18 to 32% and a hydroxypropyl content of from 7 to 15%; polyvinylpyrrolidone with a molecular weight of from 17,000 to 90,000; polyacrylic acids and polyacrylates with a molecular weight of from 400,000 to 4,000,000 and, for example, a viscosity of about 30,000 mPa·s; where the viscosity and pH values in each case relate to a 1% strength aqueous solution.

The hydratable polymers suitable according to the invention are those able in the presence of water or saliva to form a coherent viscous mass quickly, preferably within less than about 45 seconds, and more preferably about 25 seconds. Suitable natural or semisynthetic polymers are known in principle to the skilled person. Particularly suitable hydratable polymers are those which form highly viscous solutions in water, in particular nonionic polymers with a viscosity, measured as 1% strength (weight/weight) aqueous solution, of from 3 to 10,000 mPa·s, for example polyvinylpyrrolidone and cellulose ethers such as methyl cellulose, hydroxyethyl cellulose, hydroxypropyl cellulose and hydroxypropyl methyl cellulose and the like, and ionic polymers with a viscosity, measured as 1% strength (weight/weight) aqueous solution, of from 3 to 30,000 mPa·s, such as sodium carboxymethylcellulose, polyacrylic acids, polyacrylates, alginic acid, alginates, pectin, xanthan, galactomannan, guar gum, hydroxypropyl-guar gum, gelatin, gum arabic and the like, with particular preference generally being given to those with a viscosity, measured as 1% strength (weight/weight) aqueous solution, of at least about 25 mPa·s.

Whereas some of the hydratable polymers, for example polyvinylpyrrolidone or cellulose ethers, have a substantially pH-independent viscosity, a large decline in viscosity occurs in the pH range of the gastric fluid with others, for example with sodium carboxymethylcellulose, sodium alginate or polyacrylic acids. This difference can therefore be utilized to influence specifically the disintegration of the composition in the stomach. If, for example, sodium carboxymethyl cellulose, sodium alginate or a polyacrylic acid is used as hydratable polymer, on the one hand the required high viscosity in the mouth is achieved but, on the other hand, the composition rapidly disintegrates in the stomach and thus permits release of active ingredient.

If it is particularly important that a coherent, viscous mass is formed as rapidly and uniformly as possible, preferably a small particle size is chosen. As a rule, therefore, hydratable polymers with an average particle size not exceeding 200/μm, in particular not exceeding 100/μm, are preferably used.

Preferred matrix forming agents include one or more of pharmaceutical grade gelatins, pectins (nonhydrolyzed, partially hydrolyzed or hydrolyzed), hydrolyzed celluloses, either alone or in combination. Most preferred for the present invention is a gelatin matrix selected from low-bloom Type A (170-220 g), Type B (150-200 g), or a mixture of Types A and B.

Gelatin is graded and sold by its ‘Bloom Value’ that is a measurement of the strength of a gel formed by a 6 and ⅔% solution of the gelatin that has been kept in a constant temperature bath at 10 degrees centigrade (50° F.) for 18 hours. A texture analyzer is then used to measure the weight in grams that is required to depress a standard AOAC plunger 4 millimeters into the gel. If this procedure requires 200 grams, then the gelatin is a 200-bloom gelatin. A lower bloom value produces a weaker gelatin. The three most common grades of gelatin are 125, 175 and 250 although other grades maybe used in this invention.

Other functional characteristics of gelatin can be summarized as follows: natural gelling, thickening, stabilizing, foaming, water binding, whipping, and emulsifying. A variety of different textures, hard or soft, short or long, can be obtained by simply changing the concentration and/or Bloom strength of the gelatin. Among the many parameters to consider during the selection process in addition to Bloom Value are firmness, relaxation, swelling, adhesiveness, tack, stickiness, cohesiveness, rupture/burst and extensibility.

Polymeric matrices (gelatin) can have two isoelectric points, depending on the method of preparation. So-called Type A gelatin, derived from an acid-treated precursor, has an isoelectric point of between pH 7 and 9. Type B gelatin, obtained from an alkali-treated precursor, has an isoelectric point of approximately pH 5. Type A gelatin acts best as an emulsifier around pH 3, where it is positively charged. On the other hand, Type B gelatin is best around pH 8, where it is negatively charged. Both Type A and Type B gelatin can be used in this invention. To avoid an incompatibility, all emulsifying agents should carry the same charge.

The gelation temperature or melting point of gelatin-water systems is in the range of 20 to 40 ° C. The gelation temperature increases with increasing gelatin content and with increasing gelatin molecular weight, as does the solution viscosity. Below the gelation temperature, the gel rigidity increases with increasing gelatin content. While the modulus and the ultimate strength of aqueous gels increase with increasing gelatin content, the elongation at break is not much affected. Gel strength and rigidity are highest at the isoelectric point, where cross-linking by salt bridges is most extensive. While typical aqueous gelatin gels contain 20 to 45% solids (polymeric matrix), at room temperature pectin and agar form strong gels, which contain only 1 to 4% solids. For use in this invention, the percent of polymeric matrix may range from 1 to 75%.

Besides the chemical nature of polymeric matrix and solvent (usually aqueous), the three most important factors influencing the gelling of polymer solutions are concentration, temperature, and molecular weight. Lower temperatures, higher concentrations of gelling polymer, and higher molecular weights of gelling materials promote gelling and produce stronger gels.

For a typical gelatin, 10% solutions (solutions containing 10% polymeric matrix) begin to gel at about 25° C.; 20% solutions at about 30° C.; and 30% solutions at about 32° C. With some polymeric matrices, the gelation is reversible; the gels liquefy when heated above these temperatures. Gelation is rarely observed above 34° C. regardless of concentration, so that gelatin solutions do not gel at 37° C. The gelation temperature or gel point is highest at the isoelectric point, where the attachment between different chains by coulombic attraction or ionic bonds between carboxylate groups and alkylammonium, guanidinium or imidazolium groups is most extensive.

The gelation temperature or the melting point of the gel depends more strongly on temperature and concentration than on pH. The combination of an acid pH considerably below the isoelectric point and a temperature of 37° C. completely prevents the gelation of gelatin solutions. Agar and pectic acid solutions set to gels at only a few percent of solids. Unlike most water-soluble polymers, methylcellulose, hydroxypropylcellulose, and polyethylene oxide are more soluble in cold that in hot water. Their solutions therefore tend to gel on heating.

The stiffeners may include substances to adjust viscosity of the formulation so that it remains flexible but doesn't substantially change shape. Examples of suitable substances include gelatins (different from that of the matrix), alkylcelluloses, sugar alcohols, starches, alginates, hydrogels, and polyvinyl alcohols. These stiffening agents are selected and provided in an amount that enhances the stability of the formulation relative to a corresponding formulation without the stiffening agents. Ideally, the resulting formulation is stable under refrigeration at about 3-8° C. for one to two years minimally, or at room temperature preferably for one to two years.

Excipients or other agents that may enhance the physical properties of the composition to aid swallowing or preserve the activity of the active ingredient(s) may be included alone or in combinations. Example of excipients useful in the present invention include preservatives, olfactory stimulants, salivation stimulants, solubilizing agents, pH modification agents, sweeteners, flavoring agents, antioxidants, flavors, surfactants, colorants, lubricating or viscous mineral or vegetable oils, natural or synthetic plant extracts such as aromatic oils, and humectants and viscosity enhancers such as glycerol, propylene glycol, sorbitol or glycerin, may also be included to provide additional viscosity, plasticity, moisture retention and a pleasant texture and odor for the formulation.

The formulations of the ingredient may include active ingredients, such as drugs, nutraceuticals, nutrition replacement or supplements, diagnostic compounds, biomarkers, and radiolabeled drugs. The active ingredient(s), alone or in combination with other active ingredients, may include pharmaceuticals agents (over-the-counter, prescription, or new chemical entities (NCE)), vitamins, minerals, and diagnostics or any other biologically active agent or health supplement that is normally administered via swallowing. Examples of pharmaceutical agents that may be incorporated in the gelatinous composition are acetaminophen, captopril, diltiazem, nifedipine, dicyclomine, alprazolam, amitriptyline, clomipramine, propranolol hydrochloride, labetalol, allopurinol, metformin, atenolol, potassium chloride, lithium, levothyroxine sodium, ibuprofen, estrogen, and acetyl salicylic acid. However, substantially any pharmaceutical agent or biologically active agent or combination of biologically active agents may be used as the active ingredient, either by adding the active agent(s) to the mixture to be jellied or by adding solutions, emulsions, liposomes, or complexes of the active agent to the mixture to be jellied.

Other dosage forms such as pellets, beads, mini-tabs which contain one or more active ingredients may be incorporated into the delivery device. These other dosage forms may serve to physically separate multiple active ingredients so as to prevent degradation, to allow a modified release of one or more active ingredients, to allow multiple modified release patterns of one or more active ingredients, to allow different modified release patterns of different active ingredients, to allow modification of the local pH for one active ingredient in the presence or absence of another active ingredient, to modify the local region where absorption of medicament takes place or to provide additional barriers for taste masking and for protection from degradation.

Pharmaceutical Formulations Having a Segmented Shape which Facilitates Dose Adjustments

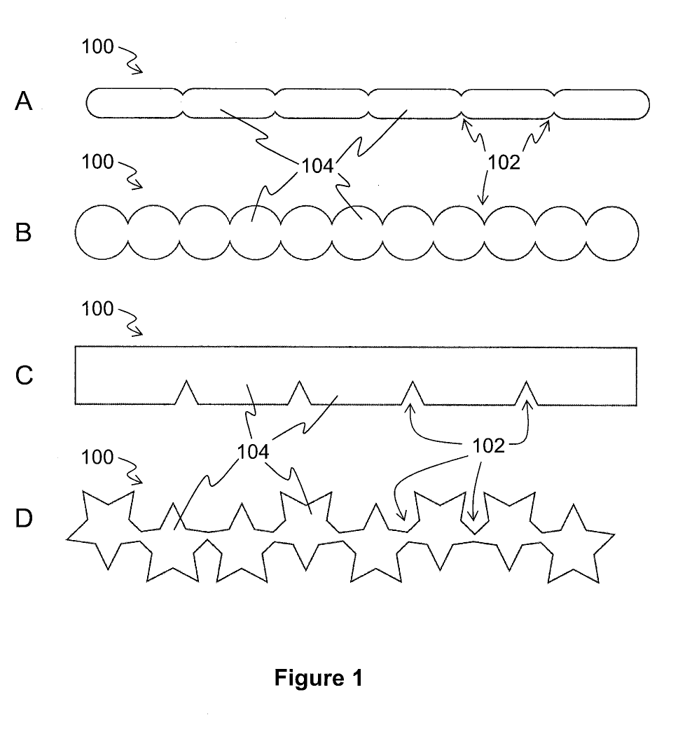

FIGS. 1A-D illustrate various examples of delivery devices according to the invention. The figures show flexible dosage form including segmented dosage units that may be broken apart to provide a required patient dose of the active ingredient. Each segment includes a predetermined amount of one or more active pharmaceutical ingredients. The dosage units may be provided in a wide variety of shapes. For example, FIG. 1A shows a dosage form with generally cylindrical segments 104 demarcated by constrictions 102 which facilitate breaking of the dosage form into a patient dose. FIG. 1B shows a similar shape in which the segments 104 are generally spherical. FIG. 1C shows a generally flat or tape-shaped formulation having indentations 102 for breaking off a patient dose. FIG. 1D shows an embodiment in which the units are a novelty shape, such as a star, animal, cartoon character or the like. In some embodiments, to facilitate ease of swallowing, the dosage form is about ⅜ inch in diameter. Segments may be broken or cut apart and ingested individually or while still connected together.

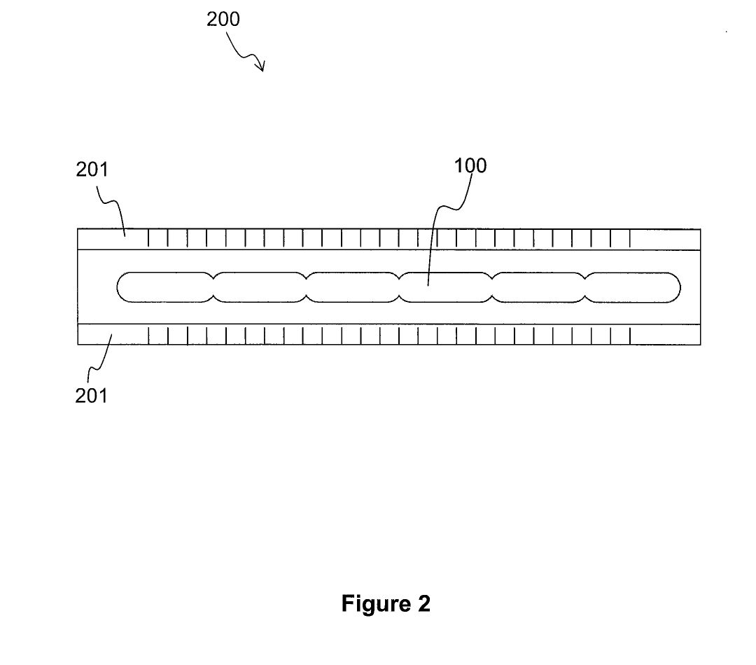

In some embodiments, the flexible delivery device 100 may be provided with or mounted on a substrate 200, as shown in FIG. 2. Dosage units may, for example, be determined using a ruler with demarcations 201 for dose, age, weight, pain scale, or any other factor affecting dosing.

In an alternative embodiment, by reducing the amount of gel base, the drug may be delivered as a paste. The paste may, for example, be administered with a metering gun for precise drug delivery in pediatric or veterinary populations.

Pharmaceutical Formulations that Mask the Taste of Active Pharmaceutical Ingredients

Another embodiment of the invention includes coatings which prevent contact with surface of the mouth. The drug can be coated and the coated drug particles can be included in the formulations of the invention. Typically particles would have a size that ranges from a few microns to a few centimeters in diameter.

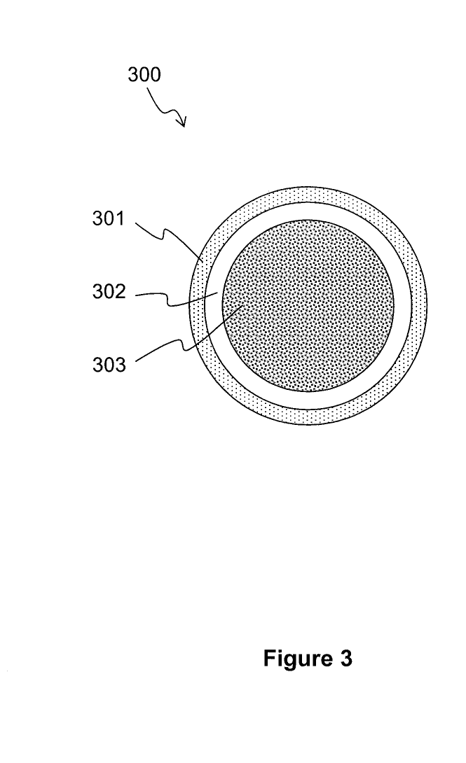

In one embodiment, illustrated in FIG. 3, includes lipid or hydrophobic polymer coating 301 covers the outside of the formulation, thereby masking the taste of the active ingredients and rendering the formulation impervious to external erosion. A second coating 302 includes the active ingredients, which are coated onto an inactive core 303. This approach prevents exposure to the taste buds, and protects the drug substance from eroding agents. It will be appreciated that a wide variety of techniques are available for forming coated drug particles. The invention includes flexible dosage forms including such coated particles therein.

Pharmaceutical Formulations that Facilitate the Delivery of Poorly Soluble Drugs

Coating technologies can also be used to render hydrophilic particles including lipophilic drugs, and the hydrophilic particles can be included in the flexible delivery devices of the invention.

The drug delivery compositions of the invention may also be useful for delivery of relatively insoluble compounds. An internal liposome, with or without a surfactant, may also be employed to enhance presentation of the drug. Other agents may be included, such as: one or more agents that improve intestinal drug absorption, such as: cremophor or bile salts; one or more agents that inhibits intestinal drug degradation, such as: buffer salts, anti-oxidants and anti-microbials; and/or one or more agents agent targeted to the enteric system, such as cellulose acetate pthallate.

Erosion of the enterocoating permits synchronous release of the drug and the intestinal drug absorption agent and/or the agent which inhibits intestinal drug degradation.

In an alternative approach, the active ingredient can be included in micelles within the flexible delivery devices of the invention. Micelles can be created by adding a surfactant to the gelatin mix while it is being formed. The micelles will form inside the delivery device and capture the active ingredient. This approach is particularly helpful for delivery of lipophilic active ingredients.

In another alternative approach, the flexible delivery device of the invention can be provided with pockets of oil (e.g., olive oil) therein which contain a lipophilic active ingredient therein. When the delivery device dissolves in the intestine, the oil will present the active ingredients to the enterocytes of the intestinal wall. Any pharmaceutical grade oil would be suitable.

In another embodiment, the flexible delivery device includes a water soluble drug in an aqueous carrier that is coated with an oil. This approach has the advantage that the oil at the surface of the dosage form may prevent the drug from migrating to the tongue, permitting the subject to ingest the delivery device without tasting the active ingredient.

Manufacture

Typically, a hydratable polymeric matrix material is mixed with water or other appropriate solvent to form a suspension, into which one or more active ingredient(s) and optionally one or more stiffeners or excipients are blended. The mixture is then processed to induce gelling, e.g., heating or cooling depending upon the polymeric matrix. The mixture is then cast into molds wherein it gels. Alternatively, the mixture is allowed to cool and the gel is extruded as the dosage form from the mold. The product may be molded and packaged, e.g., with “blow, fill and seal” technology similar to that used currently in sterile product manufacturing.

Administration

The delivery devices of the invention are administered to a subject orally, e.g., by ingestion through the mouth, buccal delivery, or sublingual delivery. The segmented delivery devices may be broken apart or cut to provide a desired dosage, which is ingested by the subject. A string of several segments may be further broken apart prior to ingestion, or may be ingested together.

### EXAMPLES

### Examples 1

### Ibuprofen Dosage Form

| | | |
|---|---|---|
| Gelatin | 5 | g |
| Water | 32.5 | ml |
| Ibuprofen | 12.5 | g |

The gelatin is dissolved in the water and the solution is heated at 40-50° C. for 10 minutes. The ibuprofen is mixed with the solution and the mixture is heated for another 5 min. The mixture is then cast into molds and allowed to cool for 5 hours, after which the dosage forms are removed from the molds and packaged.

### Example 2

### Ibuprofen Dosage Form

| | | |
|---|---|---|
| Gelatin | 5 | g |
| Water | 30 | ml |
| Ibuprofen | 30 | g |
| Excipients (flavoring agent, preservative, and anti-oxidant) | 2 | g |

The gelatin is dissolved in the water and the solution is heated at 40-50° C. for 10 minutes. The ibuprofen and excipients are mixed with the solution and the mixture is heated for another 5 min. The mixture is then cast into molds and allowed to cool for 5 hours, after which the dosage forms are removed from the molds and packaged.

### Example 3

### NCE Dosage Form

| | | |
|---|---|---|
| Gelatin | 2 | g |
| Water | 50 | ml |
| Active ingredient | 3 | g |
| Excipients (olfactory agent and preservative) | 5 | g |

The gelatin is dissolved in the water and the solution is heated at 40-50° C. for 10 minutes. The active ingredient may be any pharmaceutical agent amenable to oral administration. The active ingredient and excipients are mixed with the solution and the mixture is heated for another 10 min. The mixture is then cast into molds and allowed to cool for 3 hours, after which the dosage forms are removed from the molds and packaged.

### Example 4

### NCE Dosage Form

| | | |
|---|---|---|
| Gelatin | 2 | g |
| Water | 50 | ml |
| Active ingredient | 20 | g |

The gelatin is dissolved in the water and the solution is heated at 40-50° C. for 10 minutes. The active ingredient is mixed with the solution and the mixture is heated for another 10 min. The mixture is then cast into molds and allowed to cool for 3 hours, after which the dosage forms are removed from the molds and packaged.

### Example 5

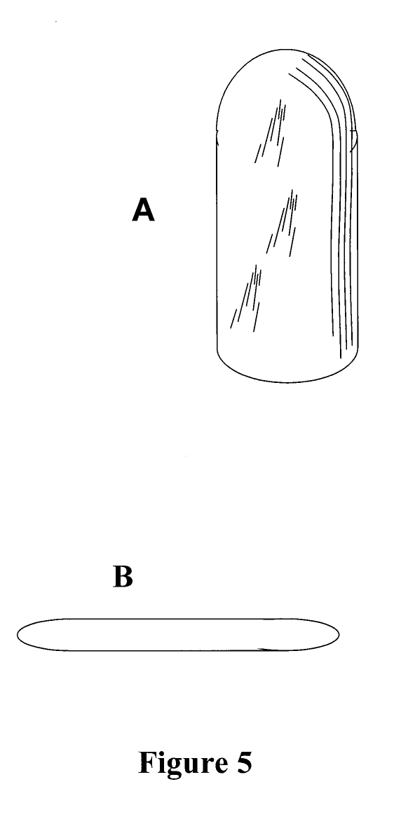

26 subjects received and congested eight (8) different combinations of shaped FIGS. 5A bullet shaped and 5B worm shaped and weighted capsules comprising the following:

| Shape | Weight |
|---|---|
| 5 × 700 mm Worm shaped | 1.4 g |
| 6 × 700 mm Worm shaped | 2.0 g |
| 7 × 700 mm Worm shaped | 2.4 g |
| 10 × 350 mm Worm shaped | 2.7 g |
| Bullet | 1.4 g |
| Bullet | 1.45 g |
| Bullet | 2.1 g |
| Bullet | 2.3 g |

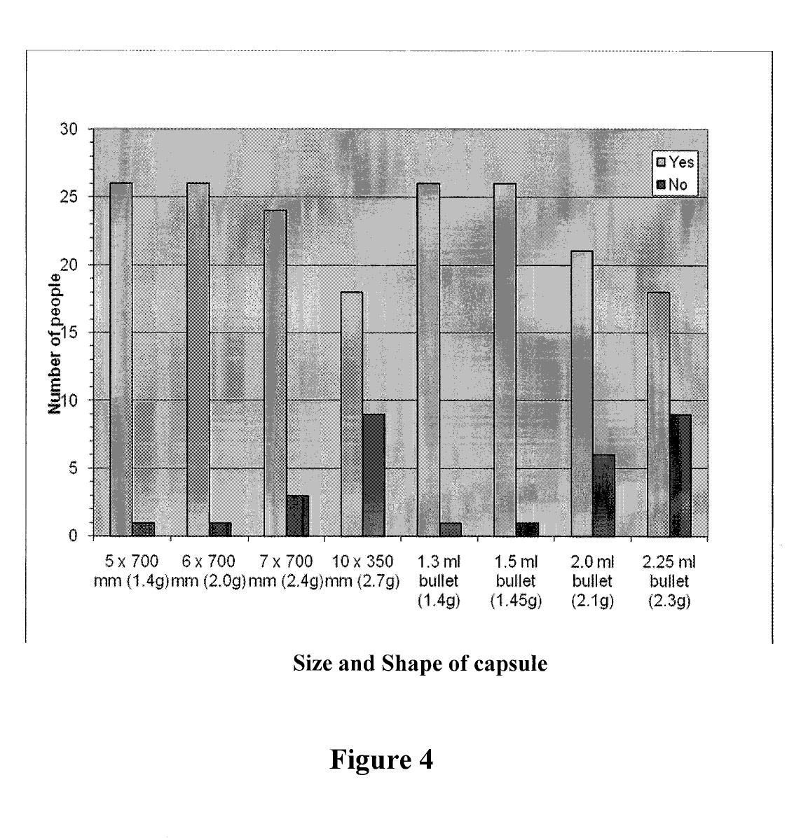

The shaped forms were fabricated from 5-20% gelatin (type A, 225 bloom) as the gelling agent. Of the 26 subjects enrolled in the study, at least 60% found the capsules of the present invention to exhibit increase swallowability as shown in FIG. 4. Clearly there are some optimal sizes and shapes. Notably, because of the ease of swallowability, patients using such delivery devices are less likely to stop taking a therapeutic agent impregnated into the hydrated polymeric matrix of the present invention.

That which is claimed is:

1. A swallowable delivery device for administering an active agent to subject having difficulty swallowing, the device comprising:
a gelatinous matrix component;
a stiffening agent; and
an active agent, wherein the active agent and stiffening agent are dispersed in the gelatinous matrix component to form a formulation for fabricating into a shaped form for easy swallowability.

2. The swallowable delivery device of claim 1, wherein the shaped form is a segmented form in which each segment includes a predetermined amount of the active agent.

3. The swallowable delivery device of claim 1, wherein the shaped form is an unsegmented elongated form in which the active agent is evenly distributed throughout the form, such that a unit dosage can be obtained by measuring the length of the unsegmented elongated form and removing a section to provide the unit dosage form.

4. The swallowable delivery device of claim 1, further comprising a taste masking coating on an outer surface thereof.

5. The swallowable delivery device of claim 1, wherein the active agent is poorly soluble in the gelatinous matrix component and is provided in a liposome.

6. The swallowable delivery device of claim 1, wherein the active agent comprises drugs, nutraceuticals, nutrition replacement, nutrition supplements, diagnostic compounds, biomarkers, or radiolabeled drugs.

7. The swallowable delivery device of claim 1, wherein the gelatinous matrix component is pharmaceutical grade gelatin, pectins, hydrolyzed celluloses or a combination thereof.

8. The swallowable delivery device of claim 1, wherein the gelatinous matrix component is, selected from low-bloom Type A (170-220 g), Type B (150-200 g), or a mixture of Types A and B.

9. The swallowable delivery device of claim 1, wherein the stiffener component is a gelatin which is different from that of the matrix component, alkylcellulose which is different from the matrix component, sugar alcohols, starches, alginates, hydrogels, or polyvinyl alcohols.

10. The swallowable delivery device of claim 10, wherein the stiffener component enhances the stability of the formulation relative to a corresponding formulation without the stiffening agents.

11. The swallowable delivery device of claim 10, wherein the formulation is stable at room temperature for at least one year.

12. The swallowable delivery device of claim 1, wherein the active agent is encapsulated into pellets, beads, or mini-tabs and dispersed throughout the gelatinous matrix component.

13. The swallowable delivery device of claim 12, wherein at lease two active agents are encapsulated into pellets, beads, or mini-tabs and dispersed throughout the gelatinous matrix component and wherein the active agents are physically separated from each other by the encapsulation.

14. The swallowable delivery device of claim 2, wherein the segment has a shaped form is cylindrical, spherical, worm shaped, bullet shaped or tape shaped.

15. The swallowable delivery device of claim 1, wherein the gelatinous matrix component is pharmaceutical gelatin in a 10% to 20% solution.

16. A method of administering an active therapeutic agent to a subject, the method comprising:
providing swallowable delivery device comprising:
a gelatinous matrix component;
a stiffening agent; and
an active agent, wherein the active agent and stiffening agent are dispersed in the gelatinous matrix component to form a formulation for fabricating into a shaped form for easy swallowability; and
ingesting the shaped form.

17. The method of claim 16, wherein the active agent comprises drugs, nutraceuticals, nutrition replacement, nutrition supplements, diagnostic compounds, biomarkers, or radiolabeled drugs.

18. The method of claim 16, wherein the gelatinous matrix component is pharmaceutical grade gelatin, pectins, hydrolyzed celluloses or a combination thereof.

19. The method of claim 16, wherein the stiffener component is a gelatin which is different from that of the matrix component, alkylcellulose which is different from the matrix component, sugar alcohols, starches, alginates, hydrogels, or polyvinyl alcohols.

20. The method of claim 16, wherein at lease two active agents are encapsulated into pellets, beads, or mini-tabs and dispersed throughout the gelatinous matrix component and wherein the active agents are physically separated from each other by the encapsulation.

21. The method of claim 16 wherein the shaped form is cylindrical, spherical, worm shaped, bullet shaped or tape shaped.

22. The method of claim 16, wherein the gelatinous matrix component is pharmaceutical gelatin in a 10% to 20% solution.

23. The method of claim 16, wherein the delivery device is a segmented shaped form which includes multiple unit dosages.

24. The method of claim 23, further comprising breaking or cutting off one or more segments from the segmented shaped form to provide a unit dose.

25. A kit comprising multiple oral delivery devices for administration of an active therapeutic agent, wherein the oral delivery devices comprises:
a gelatinous matrix component;
a stiffening agent; and
an active agent, wherein the active agent and stiffening agent are dispersed in the gelatinous matrix component to form a formulation for fabricating into a shaped form for easy swallowability.

26. A swallowable delivery device in a shaped form for administering an active agent to subject having difficulty swallowing, the device comprising:
a inactive core comprising a gelatinous matrix component;
a coating positioned on the inactive core, wherein the first coating comprises:
a gelatinous matrix component; and
an active agent, wherein the active agent and stiffening agent are dispersed in the gelatinous matrix component; and
an outside coating to mask the taste of the active ingredients.
